(19) United States
(12) Patent Application Publication
(10) Pub. No.: US 2004/0137607 A1
(43) Pub. Date: Jul. 15, 2004

(54) Biochip cartridge

(75) Inventors: Takeo Tanaami, Tokyo (JP); Kazuhisa Fukushima, Tokyo (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: Yokogawa Electric Corporation, Tokyo (JP)

(21) Appl. No.: 10/716,417

(22) Filed: Nov. 20, 2003

(30) Foreign Application Priority Data
Jan. 9, 2003 (JP) 2003-002813
Jan. 20, 2003 (JP) 2003-010486
Jan. 20, 2003 (JP) 2003-010487

(51) Int. Cl. C12M 1/34

(52) U.S. Cl. 435/287.2; 435/288.5

(57) ABSTRACT

The present invention provides a biochip cartridge wherein an elastic body is used for a substrate member in order to stabilize the feeding of blood or solution and whereby it is possible to avoid the risk of accidental contact of the operator with solutions due to mishandling.

The biochip cartridge comprises a tabular substrate member formed using an elastic material and a flexible cover air tightly attached to the surface of the substrate member, wherein at least an area for storing biopolymers, an area for detecting desired biopolymers from the biopolymers that have been preprocessed, and a flow path for connecting the areas is formed on the substrate member, so that biopolymers can be successively moved from the biopolymer storage area to the biopolymer detection area through the flow path.

### BACKGROUND OF THE INVENTION

1. Field of the Invention

The present invention relates to a biochip cartridge for use with biochips intended to test biopolymers, such as DNA, RNA (mRNA or cDNA, for example) and protein and, more particularly, to a biochip cartridge that is extremely safe and can reduce the cost of testing.

2. Description of the Prior Art

Biochip cartridges for testing DNA or other biopolymers have been well known. For example, a biochip cartridge (also simply referred to as biochip) used to read the sequence of a DNA target by scanning a hybridized DNA chip with a biochip reader as illustrated in FIG. 1 is described in the Japanese Laid-open Patent Application 2001-235468.

In this biochip, excitation light is radiated at the hybridized DNA chip within biochip 10 and fluorescent light emitted from a fluorescent marker is read using biochip reader 20 so that the sequence of the DNA target, for example, is identified. It should be noted that cartridge 11 is formed using a material that is transparent to the excitation light and fluorescent light.

Biochip 10 mentioned above is configured in such a manner that substrate 12, on which a multitude of sites CL of the DNA probe chip are arranged in arrays, is housed within cartridge 11 as illustrated in FIG. 2. In biochip 10, solution 15 containing target DNA segments previously marked with a fluorescent marker is injected from inlet 13 using solution infusion means 14, such as a pipette, prior to read-out operation, as shown in FIG. 3, so that the DNA segments are hybridized with the probe DNA chip.

On the other hand, such test samples as blood are sometimes found to be contaminated with a virus such as HIV. Therefore, for safety reasons there is a growing tendency to use disposable equipment for such medical appliances as syringes.

In contrast, the method of introducing a solution shown in FIG. 3 involves the risk of the operator being infected with a virus, such as HIV, as a result of accidental contact with the solution due to mishandling. This risk exists because the method always involves transferring the solution from solution infusion means 14 to cartridge 11.

Another problem with the conventional biochip is that the cost of testing increases since more than one kind of medical equipment must be disposed of, including syringes, appliances used for preprocessing purposes, injection means, DNA chips, and so on.

The biochip illustrated in FIG. 4 has solved the aforementioned problems. The biochip comprises blood collection tube 31, instead of a conventional conical tube, which is inserted into a syringe cylinder in order to collect blood. The blood collection tube is formed into a cylindrical shape using a solid material transparent to excitation light and fluorescent light produced. The opening of blood collection tube 31 is sealed with a rubber plug 32 which is pierced through the middle with a needle, and blood collection tube 31 as a whole is kept under negative pressure.

Blood collected through the needle is temporarily retained within collection block 33 and then introduced to preprocessing block 34, where the blood is preprocessed. This preprocessing comprises a series of processes, including separating lymphocytes from the blood, isolating DNA from the separated lymphocytes, and adding a fluorescent marker to the isolated DNA.

Housed in the innermost section of blood collection tube 31 is substrate 35, similar to the one shown in FIG. 1, on which probe DNA segments are arranged in arrays. In the innermost section, DNA segments that infiltrate from preprocessing block 34 and the probe DNA segments are hybridized.

Although such a biochip cartridge as described above is advantageous in that processes, including blood collection, preprocessing and hybridization, are performed consistently and automatically, the cartridge requires a rigid blood collection tube and is therefore expensive. Furthermore, the biochip cartridge involves using an air suction pump or the like to produce negative pressure, and thus overall costs are comparatively high.

A biochip that has solved the aforementioned problems is described in the Japanese Laid-open Patent Application 2002-365299 submitted by the applicant of the application concerned. This biochip is configured in such a manner as illustrated in FIG. 5. The abovementioned biochip, which is indicated by 40 in FIG. 5, has good flexibility and is formed into a flat, airtight bag-like shape, using a material transparent to fluorescent light and excitation light.

Blood collection bag 41 has a rectangular outline, as shown in the plan view of FIG. 5(b), and the periphery of the bag is sealed airtightly. The middle area of the bag is shaped like a fish. The bag's opening, which corresponds to the mouth of a fish, is closed airtight with plug 42. Plug 42 is formed using a rubber-like material and a syringe needle is pierced through plug 42 at the time of blood collection. When the syringe needle is pulled out after blood collection, the pinhole thus opened immediately closes, preventing the collected blood from leaking out of the biochip.

In sequence from plug 42 to the innermost section of the biochip, collection block 43, preprocessing block 44, combination block 45, and waste liquid reservoir 47 are formed in blood collection bag 41.

Collected blood is stored in collection block 43. Hooks 43a and 43b are formed on both the top and bottom sides of the jacket of collection block 43. At the time of blood collection, collection block 43 is expanded by pulling outwards engagement members engaged with these hooks 43a and 43b.

In preprocessing block 44, a process of isolating targets of interest from the collected blood is executed. Combination block 45 is provided with substrate member 46, on which a plurality of probes (herein assumed to be DNA) are arranged in arrays, so that targets isolated in preprocessing block 44 can be combined complementarily with the probes.

Waste liquid reservoir 47 is a pocket provided in order to retain an unnecessary solution forcibly driven out of preprocessing block 44 and combination block 45. The pocket is compressed in its initial state.

Pockets 48 and 50 corresponding to the dorsal and abdominal fins of a fish are formed on the sides of preprocessing block 44 opposing each other. Solutions necessary to isolate targets (DNA, RNA or protein) from blood are encapsulated in pockets 48 and 50, respectively.

Plug valves 49 and 51 serving as bulkheads are formed in junctions (narrow passages) between pocket 48 and preprocessing block 44 and between pocket 50 and preprocessing block 44. These valves are designed to break when the pressure of solutions within the pockets rises to a given level.

After the collected blood is stored in the collection block 43 of blood collection bag 41, blood collection bag 41 is pinched between rollers 61 and 62 that rotate as shown in FIG. 6, so that the bag is squeezed in the direction from collection block 43 toward preprocessing block 44.

The axial length of rollers 61 and 62 is made to be greater than the width of blood collection bag 41, so that the rollers uniformly apply pressure to the entire width of blood collection bag 41.

As rollers 61 and 62 rotate, the collected blood is forced to move toward preprocessing block 44.

When rollers 61 and 62 advance and begin squeezing pocket 48, the internal pressure thereof rises and therefore plug valve 49 breaks. When plug valve 49 breaks, a solution within pocket 48 flows into preprocessing block 44, where a given process based on the solution is executed.

Then, when pocket 50 is also squeezed by rollers 61 and 62, plug valve 51 likewise breaks and a solution within pocket 50 flows into preprocessing block 44, where a given process is executed.

Consequently, it is possible to easily submit blood collection bag 41 to time-differentiated processing by displacing the mounting positions of the pockets from each other. In other words, it is possible to submit the bag to the process of separating lymphocytes from blood and isolating DNA from the lymphocytes thus separated and the process of, for example, adding a fluorescent marker to the isolated DNA, with a time difference provided between these processes.

When the process in preprocessing block 44 is completed, then rollers 61 and 62 are rotated further. This operation feeds the preprocessed blood toward combination block 45, where hybridization with probe DNA chips arranged on substrate member 46 takes place.

It should be noted that extra amounts of blood and solution forcibly driven out of preprocessing block 44 accumulate in waste liquid reservoir 47.

DNA chips that have undergone hybridization are read in the same way as the conventional method, using a biochip reader (not shown in the figure).

As described heretofore, processes from blood collection to preprocessing and hybridization are executed consistently within a hermetically sealed blood collection bag. Therefore, it is possible to prevent accidental contact of the operator with solutions due to mishandling. In addition, since such a blood collection bag as described above can be easily fabricated using a flexible inexpensive material, it is possible to easily realize an inexpensive biochip.

However, such a conventional biochip as discussed above has had the following problems:

(1) Blood collection bag 41 is squeezed unevenly when being pinched and forwarded by rollers 61 and 62, thus hindering the flow of blood or stabilization of solution.

(2) Since blood collection bag 41 is soft, self-fluorescence tends to occur easily due to the adhesive agent, coatings or plastic materials used therein. This self-fluorescence constitutes background noise and causes the S/N ratio to change. Consequently, it becomes infeasible to detect weak fluorescent light (signal) emitted from the fluorescent substance with which DNA has been marked.

(3) It is not possible to store mRNA or DNA as is, without submitting it to hybridization, nor is it possible to make already-stored mRNA or DNA undergo hybridization only.

(4) Although the biochip in question is designed so that pre-processing and hybridization are performed within the bag and, therefore, is advantageous in that it can eliminate the risk of viruses, for example, being released from the biochip during processing, the biochip has the problem that it is not possible to use a general-purpose, slide glass type DNA microarray. Note that although there is a cassette capable of hybridization using a general-purpose, slide glass type DNA microarray, the cassette requires conversion of the sample into cDNA or labeling the sample (for example, attaching fluorescent markers) in a laboratory or other places. Furthermore, use of the cassette involves post-hybridization cleaning, resulting in the problem that a specific place and special skills are required.

(5) Such a dedicated biochip as illustrated in FIG. 1 requires the use of a dedicated reader suited for that biochip, resulting in the problem that it is not possible to use general-purpose readers.

### SUMMARY OF THE INVENTION

It is an object of the present invention to solve the abovementioned problems by using an elastic body for the substrate member in order to stabilize the feeding of blood or solution and providing a biochip cartridge capable of preventing the danger of the operator accidentally coming into contact with solution due to mishandling.

It is another object of the present invention to realize a biochip cartridge that is low self-fluorescence.

It is yet another object of the present invention to provide a biochip cartridge that allows pre-processing and cleaning to be performed within the cartridge and hybridized biopolymers to be detected using a general-purpose reader.

### BRIEF DESCRIPTION OF THE DRAWINGS

FIG. 1 is a schematic view illustrating an example of a conventional biochip.

FIG. 2 is the plan view of the conventional biochip illustrated in FIG. 1.

FIG. 3 is a schematic view explaining a method of injecting a solution into the conventional biochip.

FIG. 4 is a schematic view illustrating another example of the conventional biochip.

FIG. 5 is a schematic view illustrating yet another example of the conventional biochip.

FIG. 6 is a schematic view explaining another method of handling the conventional biochip illustrated in FIG. 5.

FIG. 7 is a schematic view illustrating one embodiment of the biochip cartridge in accordance with the present invention.

FIG. 8 is a schematic view explaining the behavior of the biochip cartridge illustrated in FIG. 7.

FIG. 9 is a schematic view illustrating another embodiment of the present invention.

FIG. 10 is a schematic view illustrating yet another embodiment of the present invention.

FIG. 11 is a schematic view illustrating yet another embodiment of the present invention.

FIG. 12 is a schematic view illustrating yet another embodiment of the present invention.

FIG. 13 is a schematic view illustrating yet another embodiment of the present invention.

FIG. 14 is a schematic view illustrating an embodiment of the separable biochip cartridge in accordance with the present invention.

FIG. 15 is a schematic view illustrating a state of the biochip cartridge being separated.

FIG. 16 is a schematic view illustrating the convex joint of the housing.

FIG. 17 is a schematic view illustrating the concave joint of the housing.

FIG. 18 is a schematic view illustrating a state of the housings being coupled with each other.

FIG. 19 is a schematic view illustrating another state of the housings being coupled with each other.

FIG. 20 is a schematic view illustrating another embodiment of the biochip cartridge in accordance with the present invention.

FIG. 21 is a schematic view illustrating another embodiment of the present invention.

FIG. 22 is a schematic view illustrating yet another embodiment of the present invention.

FIG. 23 is a schematic view illustrating another embodiment of the present invention.

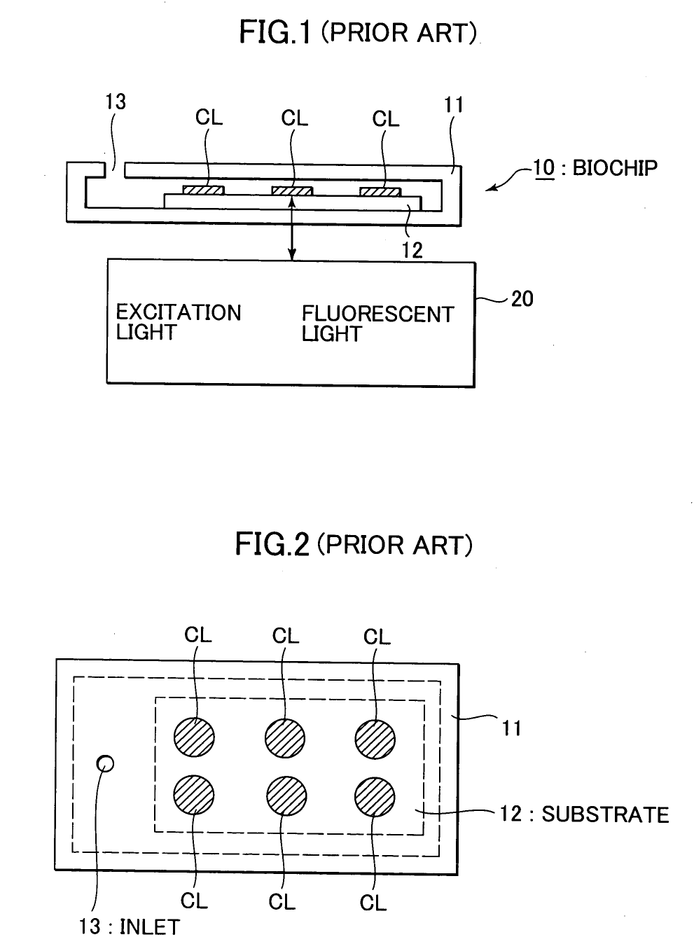

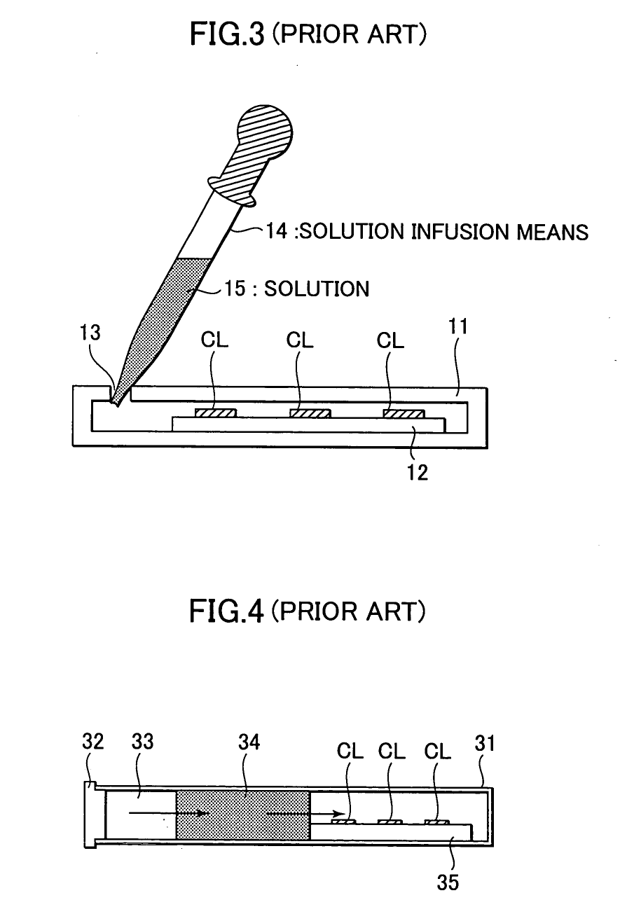

### DETAILED DESCRIPTION OF THE PREFERRED EMBODIMENTS

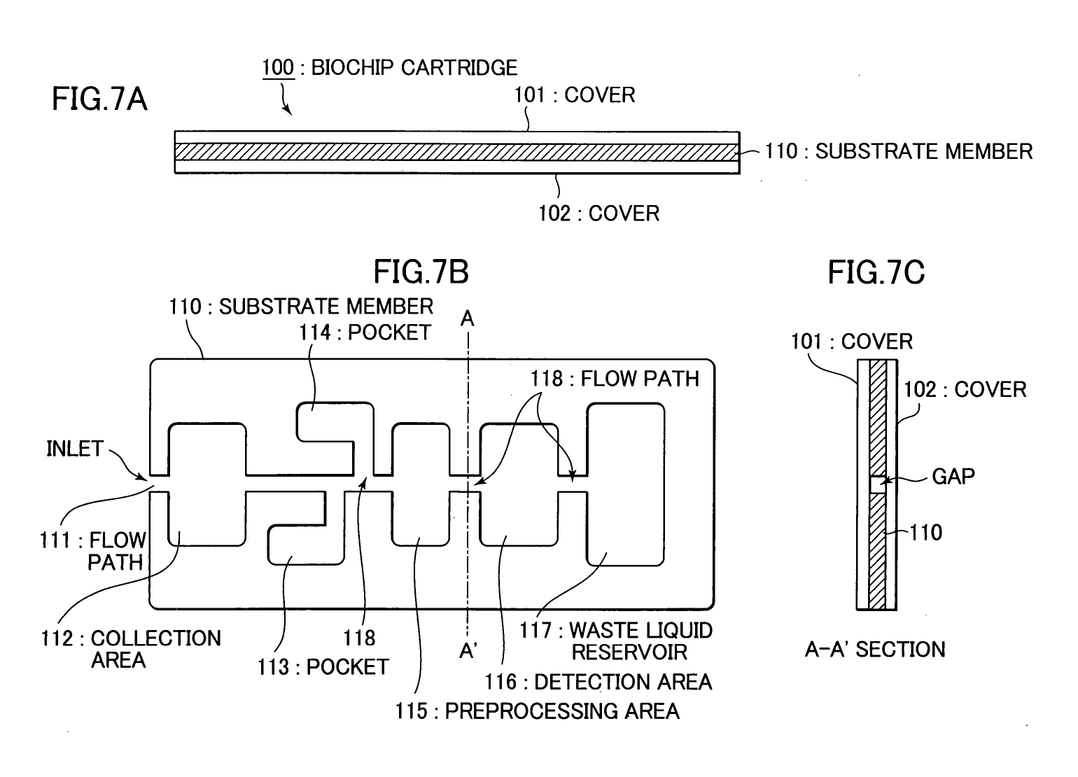

Preferred embodiments are described in detail hereinafter by referring to the accompanying drawings, wherein FIG. 7 is a schematic view illustrating one embodiment of the biochip cartridge in accordance with the present invention. FIG. 7(a) is a side view, FIG. 7(b) is the plan view of a substrate member, and FIG. 7(c) is the view of section A-A′ (including covers).

In biochip cartridge 100 illustrated in FIG. 7(a), symbols 101 and 102 indicate transparent and flexible covers made from hard plastics and symbol 110 indicates a substrate member formed using an elastic body, such as airtight, elastic rubber. Covers 101 and 102 are hermetically attached onto the top and bottom surfaces of substrate member 110 by means of, for example, adhesion.

Formed on substrate member 110 are a through-hole for flow path 111 at the inlet of the substrate member, a plurality of chambers, i.e., collection area 112, first and second pockets 113 and 114, preprocessing area 115, detection area 116 and waste liquid reservoir 117, and a through-hole for flow path 118 for connecting these elements to each other, as illustrated in FIG. 7(b).

By adhering covers 101 and 102 onto the top and bottom sides of substrate member 110, each through-hole is closed up from both the top and bottom sides. For example, flow path 118 is shaped into such an opening (hollow) as shown in FIG. 7(c).

Now each flow path and chamber are further described in detail. Flow path 111 is a sample injection inlet for injecting a solution containing a sampled biopolymer, such as blood (hereinafter simply referred to as a sample). Note that it is also possible to directly pierce a syringe needle into substrate member 110 and inject the sample into collection area 112 without providing flow path 111, by taking advantage of the fact that substrate member 110 itself is a rubber-like elastic body.

Collection area 112 is a chamber wherein a collected sample, such as blood, is stored. In first and second pockets 113 and 114, preprocessing solutions for separating a biopolymer to be detected from the sample in collection area 112 and refining and amplifying the biopolymer are stored.

In preprocessing area 115, the sample mixes with the preprocessing solutions from pockets 113 and 114 and preprocessing, such as separation, refining and amplification, is performed. Detection area 116 is a chamber provided with an array chip (not shown in the figure) onto which a biopolymer is fixed, wherein the biopolymer of the abovementioned preprocessed sample is made to complimentarily couple (hybridize) with this biopolymer so that the sample biopolymer is detected.

Waste liquid reservoir 117 is a chamber wherein waste liquid drained from detection area 116 after hybridization is stored.

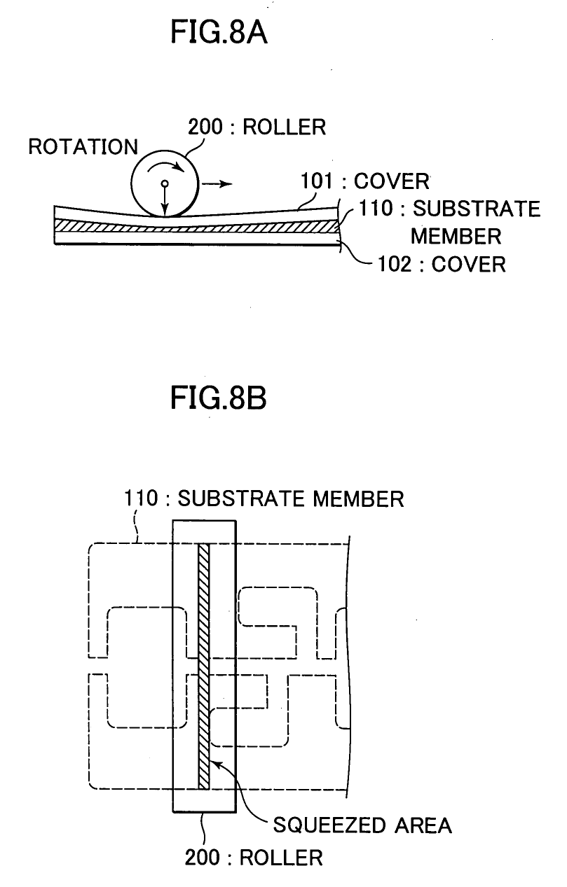

Now the usage and behavior of biochip cartridge 100 configured as discussed above is described. After a sample is injected into collection area 112, biochip cartridge 100 is placed on a flat plate (not shown in the figure), for example, and cylindrical roller-like rigid body 200 (hereinafter simply referred to as roller 200) is levelly pressed onto cover 101, as illustrated in FIG. 8(a) and rolled from the inlet side toward preprocessing area 115.

Cover 101 is deformed as roller 200 is pressed down and substrate member 110 is squeezed. As a result, flow path 111 immediately below the center of roller 200 is narrowed down and closed up, thus forming a temporary valve. As roller 200 is rotated toward the right of the figure, the temporary valve also moves rightward. This valve has the effect of preventing the flow from reversing.

As collection area 112 is squeezed by roller 200, the sample stored in collection area 112 is driven toward the right, passes through flow path 118, and is driven out into preprocessing area 115.

Next, as pocket 113 is likewise squeezed through by roller 200, the preprocessing solution is driven through flow path 118 into preprocessing area 115. As roller 200 moves further toward the right, pocket 114 is also squeezed and a preprocessing solution stored therein is also driven into preprocessing area 115. As a result, the preprocessing solutions mix with the sample within preprocessing area 115 and preprocesses, such as biopolymer separation, refining and amplification are carried out.

It is possible to easily submit the sample to time-differentiated processing by displacing the positions of pockets 113 and 114 from each other toward the right, as illustrated in FIG. 7(b).

When preprocessing is completed, roller 200 is rotated in order to squeeze preprocessing area 115 so that the preprocessed sample is forwarded to detection area 116. In detection area 116, hybridization is carried out between the biopolymer within the sample and the biopolymer fixed on to the array chip. Biopolymers and solutions that have not undergone hybridization are sent to waste liquid reservoir 117 by rotating and moving roller 200 toward the right or by tilting the entire biochip cartridge.

The array chip for which hybridization has been carried out can be read through a transparent cover 101 using a known biochip reader (not shown in the figure).

The present invention is by no means limited to the above-described embodiments but may be embodied in other ways without departing from the spirit and essential characteristics thereof. Accordingly, it should be understood that all modifications falling within the spirit and scope of the present invention are covered by the appended claims.

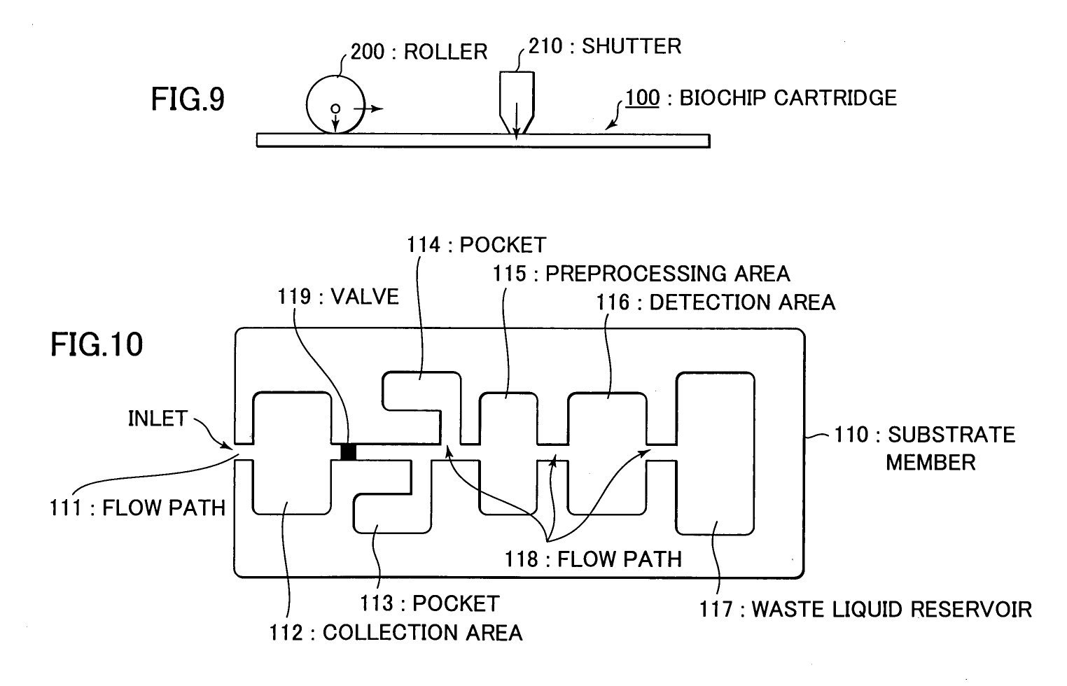

For example, it is possible to prevent the sample from inadvertently advancing further by providing the biochip cartridge with shutter 210 in addition to roller 200, as illustrated in FIG. 9, and pressing down shutter 210 and thereby blocking the flow path as necessary.

By pinching biochip cartridge 100 from top and bottom sides with two rollers, it is also possible to discharge the sample and preprocessing solutions in the same way as in the above-described embodiments.

Alternatively, the biochip cartridge can be configured by previously providing valve 119 in flow path 118 at the outlet of collection area 112, as illustrated in FIG. 10, so that the flow path is closed when injecting a sample in collection area 112 and is opened when collection area 112 is squeezed with the rollers and valve 119 is opened, thus allowing the sample to drain through flow path 118.

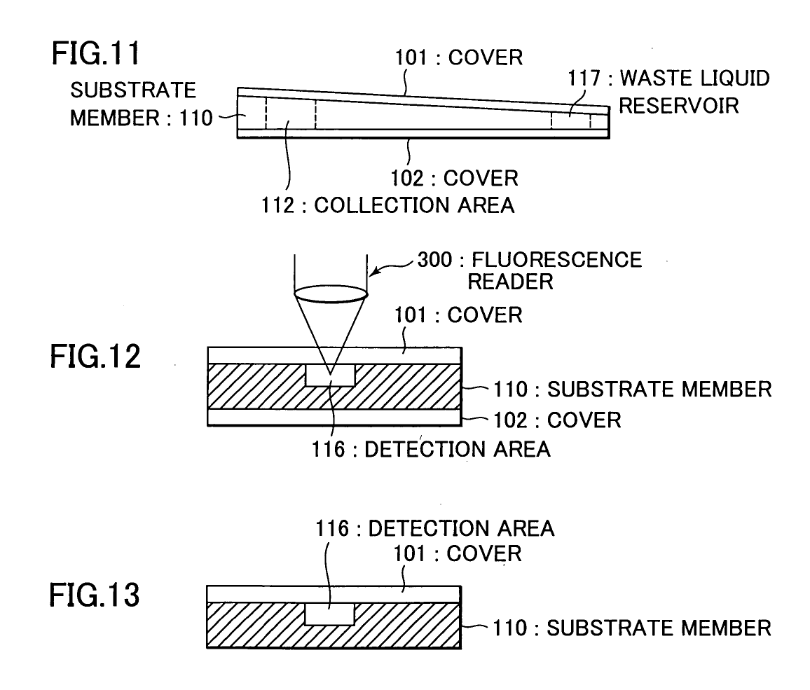

It is also possible to shape substrate member 110 into a wedge, so that the substrate member is not uniform in the thickness thereof but is thicker toward the collection area side and is thinner toward the waste liquid reservoir side, as illustrated in FIG. 11. This strategy enables the shapes of the flow path and pockets to be changed depending on the locations thereof, thus increasing the freedom of design.

It is also possible to shape the chambers and flow path of substrate member 110 into concave openings, as illustrated in FIG. 12, rather than through-holes. Furthermore, it is also possible to configure the biochip cartridge into a structure where cover 102 on the bottom side is removed, as illustrated in FIG. 13.

It is also possible to use glass or silica plates for the top and bottom side covers. Note that such transparent plates as mentioned above need not necessarily be used as long as hybridized biopolymers can be electrically detected.

It is also possible to use a gel as the substrate member. Alternatively, if the biochip cartridge is disposable, it is possible to use a plastic-deformable, unrecoverable material as the material of the substrate member.

According to the biochip cartridge configured as explained in the above-described embodiments, the following advantageous effects are provided:

(1) Processes from sample injection to hybridization are consistently carried out within a hermetically sealed biochip cartridge. Consequently, it is possible to prevent such accidents as the operator coming into contact with injected solutions due to mishandling.

(2) It is possible to easily fabricate the biochip cartridge using an inexpensive material and, therefore, an inexpensive biochip can easily be realized.

(3) Since the flow path is fixed, the amount of sample residues and the unevenness of sample squeeze are reduced, enabling the sample to be precisely discharged.

(4) Since rigid covers are used to pressurize the substrate member, self-fluorescence from the covers is extremely unlikely to occur.

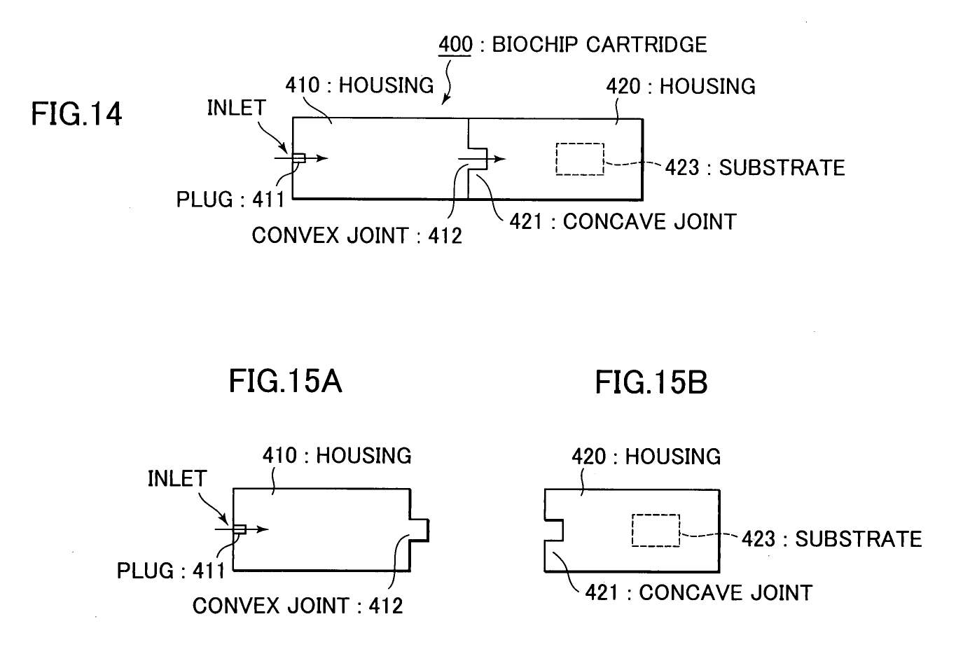

FIG. 14 is another embodiment of the present invention. The biochip cartridge as discussed in this embodiment is configured so that it is possible to eliminate the risk of accidental contact of the operator with solutions due to mishandling and to attachably and detachably separate the cartridge into two parts.

Biochip cartridge 400 illustrated in FIG. 14 is identical to biochip cartridge 100 illustrated in FIG. 7 except that biochip cartridge 400 is structured so that first housing 410 and second housing 420 are attachably and detachably separable.

Both housing 410 and housing 420 are formed using a material having good flexibility and have rectangular outlines, and the peripheries of the housings are sealed airtightly. In addition, housings 410 and 420 can be stored as separated from each other, as illustrated in FIG. 15.

Housing 410 has a rubber-like plug 411 at one end thereof and convex joint 412 at the other end thereof. Rubber-like plug 411 is airtightly mounted on the housing. A syringe needle can be pierced into plug 411 in order to inject blood containing biopolymers, such as DNA, RNA (for example, mRNA and cDNA) and protein or a homogenized biological sample into housing 410. By performing preprocessing, such as mRNA extraction from the blood, within housing 410, it is possible to extract biopolymers from the biological sample.

When the syringe needle is pulled out, the pinhole thus opened inplug 411 immediately closes, preventing the sample from leaking out of the housing.

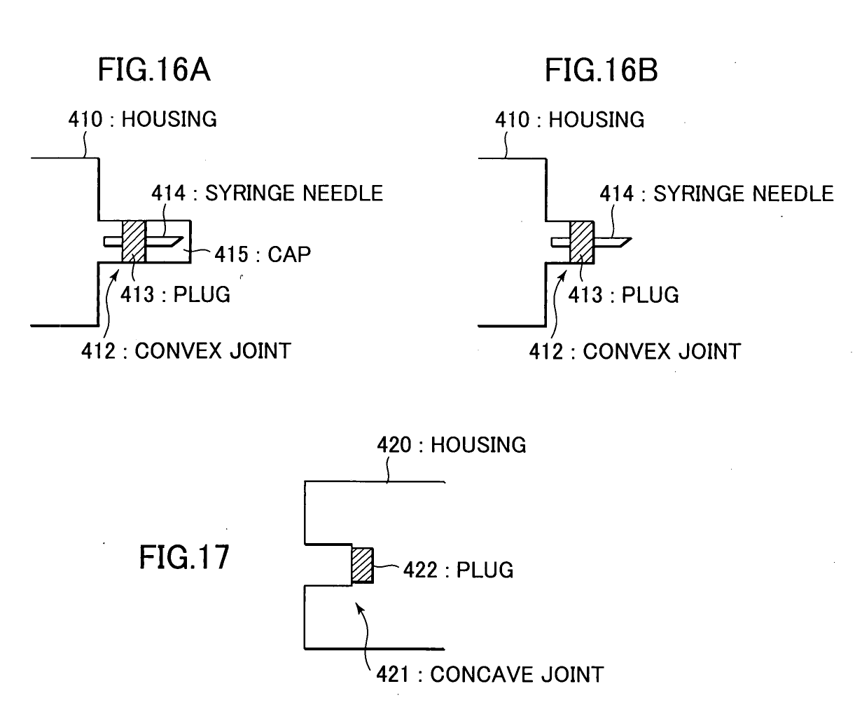

FIG. 16 is a schematic view illustrating one embodiment of convex joint 412.

Plug 413 into which a syringe needle 414 is pierced is airtightly attached to convex joint 412, and removable rubber-like cap 415 is placed on syringe needle 414. When coupling housing 410 with housing 420, cap 415 is removed as illustrated in FIG. 16(b). Housing 420 has concave joint 421 at one end thereof to couple with housing 410 and is internally provided with substrate member 423 onto which second biopolymers having sequences complementary to biopolymers (for example, mRNA) extracted using housing 410 are fixed. Note that by forming housing 420 using a transparent material, it is possible to directly read post-hybridization biopolymers using a fluorescence reader (not shown in the figure). FIG. 17 is a schematic view illustrating one embodiment of concave joint 421.

Rubber-like plug 422 into which the syringe needle of housing 410 is pierced is airtightly attached onto the bottom of concave joint 421. When the syringe needle is pulled out of plug 422, the pinhole opened by the syringe needle closes.

Now the usage of the biochip cartridge configured as described above is explained. A syringe needle is pierced into plug 411 located at the sample inlet of housing 410 and a solution containing biopolymers is injected. At this moment, cap 415 is previously placed on the convex joint 412 of housing 410, as illustrated in FIG. 16(a). By doing so, it is possible for housing 410 to temporarily store the solution with the housing 410 separated from housing 420, as illustrated in FIG. 15(a).

Since the biochip cartridge in accordance with the present invention has been made to be separable into two housings, it is possible to separately and easily inject a biological sample into housing 410 and inject biopolymers from housing 410 to housing 420 at different timings. Note that if the sample is submitted to a biopolymer extraction process as discussed above, viruses such as HIV are removed and therefore solutions drained out of housings are no longer dangerous.

When injecting a solution of biopolymers from housing 410 to housing 420, cap 415 is removed from housing 410 and convex joint 412 is inserted into the concave joint 421 of housing 420. Then, housing 410 is squeezed and the solution stored in housing 410 is fed through the syringe needle into housing 420.

After injection, housing 410 is removed from housing 420 if it is no longer necessary.

Within housing 420, hybridization is carried out between biopolymers fixed onto substrate member 423 within housing 420 and biopolymers in the solution after such necessary processing as attaching a fluorescent substance is completed. Note that the part of biochip cartridge 400 wherein substrate member 423 is located corresponds to the detection area 116 of biochip cartridge 100 illustrated in FIG. 7.

Hybridized biopolymers are detected in the same way as the method explained in the example of the conventional biochip cartridge.

It should be noted that the present invention is by no means limited to the above-described embodiments but, should be considered inclusive of the following changes and modifications:

For example, it is possible to use other means than a syringe needle in order to inject solutions into housing 410 or from housing 410 to housing 420.

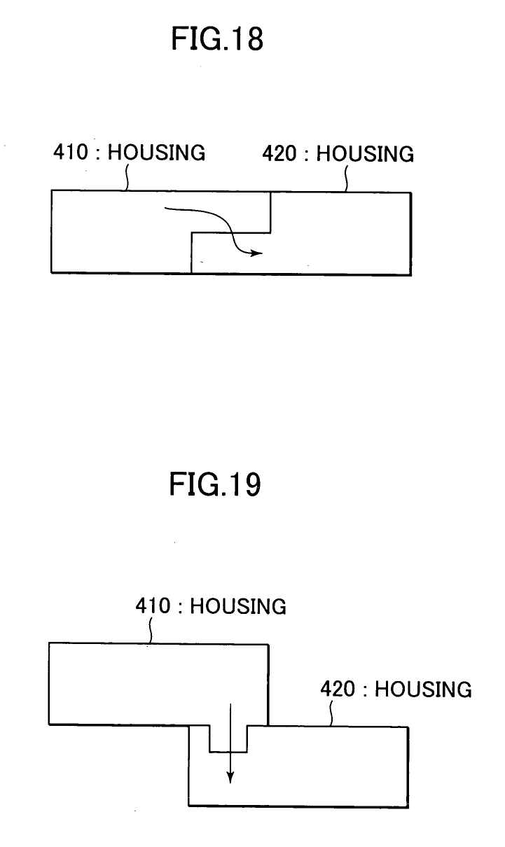

It is also possible to change the way of coupling housings 410 and 420, by cutting out the edges thereof halfway and opposite to each other so that the edges properly couple with each other, as illustrated in FIG. 18, and a solution is transferred as indicated by the arrow. It is also possible to form convex and concave joints on the sides of the housings so that the edges properly couple with each other, as illustrated in FIG. 19, and a solution is transferred as indicated by the arrow.

According to the biochip cartridge configured as explained in the above-described embodiments, the following advantageous effects are provided:

(1) The sample can be stored in a state of pre-hybridization mRNA or DNA solution in housing 410.

(2) It is easy to submit previously stored mRNA or DNA to hybridization only.

(3) Since the sample is stored in the housing in a state of mRNA solution, for example, this method of storage protects against viruses in the blood and is therefore safe.

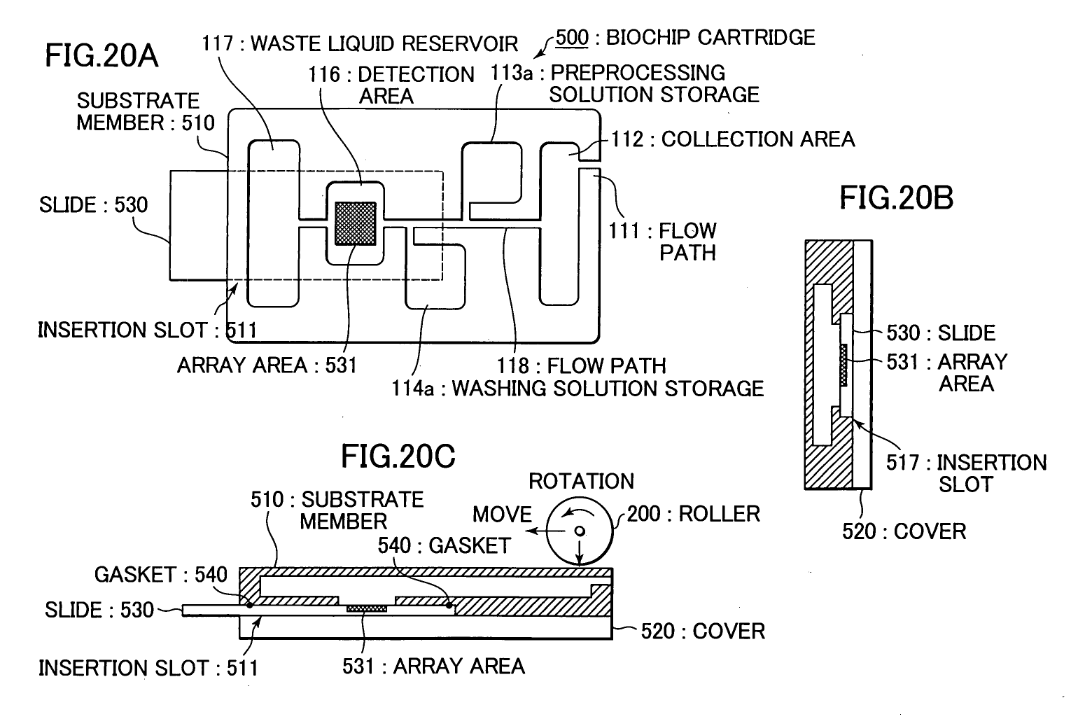

FIG. 20 is a schematic view illustrating another embodiment of the biochip cartridge in accordance with the present invention.

Biochip cartridge 500 permits preprocessing and cleaning to be carried out therewithin and hybridized biopolymers to be detected using a general-purpose reader. FIG. 20(a) is a plan view and FIGS. 20(b) and 20(c) are side views.

Biochip cartridge 500 comprises substrate member 510 and cover 520. By virtue of biochip cartridge 500, it is possible to have biological samples undergo preprocessing and hybridization in an integrated manner, with general-purpose slide glass type biopolymer microarray 530 (hereinafter simply referred to as slide 530) inserted into the biochip cartridge.

Substrate member 510 is formed using an elastic body, such as airtight elastic rubber, and a preprocessing mechanism for applying preprocessing to solutions containing biopolymers (also simply referred to as biological samples) is provided within the substrate member.

The preprocessing mechanism has a plurality of chambers comprising inlet 111 for biological samples to be injected through; collection area 112 for storing injected solutions; preprocessing solution storage 113a for storing preprocessing solution used to label biopolymers; combination area 116 (hereinafter referred to as detection area 116 since this block corresponds to the detection area illustrated in FIG. 7) for performing hybridization processes; washing solution storage 114a for storing a washing solution used to wash away (clean) a remaining extra post-hybridization biological sample; waste liquid reservoir 117 for storing the flushed extra biological sample (waste liquid); flow path 118 for connecting these constituent elements; and insertion slot 511 for slide 530 to be inserted through, as well as the capability to transfer a biological sample from the collection area to the detection area and to preprocess the biological sample in midway through the transfer in order to turn the sample into measurable biopolymers.

Cover 520 is formed using a rigid material and is airtightly joined by adhesion to the back of substrate member 510 in an attachable and detachable manner, as illustrated in FIG. 20(b).

Slide 530 has, in the center thereof, array area 531 wherein a plurality of biopolymers are fixed. Array area 531 is formed so as to be positioned immediately below detection area 116 when inserted into the insertion slot 511 of substrate member 510 or when cover 520 is temporarily removed, then attached back in place.

General-purpose slide 530 is standardized in terms of the size thereof, measuring 26×76 (mm) in Japan, 1×3 (inch) in the United States, and 25×75 (mm) in Europe.

The insertion slot of substrate member 510 is formed to the dimensions compatible with those of the slide being used. Note that these dimensions are officially prescribed in Japan by Japanese Industrial Standard JIS R3703.

In addition, gaskets 540 formed using an elastic body, such as rubber, are mounted on the bottom of substrate member 510, as illustrated in FIG. 20(c), in order to seal the boundaries between the surfaces of slide 530 and the bottom of substrate member 510. This structure makes it possible to prevent a solution within detection area 116 from leaking out.

Now the usage of the biochip cartridge configured as discussed above is described. After slide 530 is inserted into the insertion slot 511 of substrate member 510, a solution is injected into inlet 111 to fill collection area 112.

Assume at this point that preprocessing and washing solutions are previously stored in preprocessing solution storage 113a and washing solution storage 114a, respectively.

After collection area 112 is filled with the solution, roller 200 is pressed upon substrate member 510 from the top side thereof and rolled from inlet 111 toward detection area 116 (leftward), as shown in FIG. 20(c). Thus, the solution within collection area 112 is driven through flow path 118 toward detection area 116.

Next, as preprocessing solution storage 113a is squeezed by roller 200, the preprocessing solution is driven through flow path 118 toward detection area 116 and mixes with the injected solution there so that labeling is carried out.

The labeled solution hybridizes with biopolymers in the array area 531 of slide 530.

After hybridization, roller 200 is moved onward to squeeze washing solution storage 114a and causes the washing solution to discharge into detection area 116 so that biopolymers that have not undergone hybridization are washed away (cleaned) along with the solution and the waste liquid is driven into waste liquid reservoir 117.

After such cleaning, slide 530 is removed from substrate member 510 and array area 531 is measured using a general-purpose reader (not shown in the figure), in order to detect hybridized biopolymers.

By virtue of such a biochip cartridge as described above, it is possible to preprocess or clean biopolymer samples within the cartridge. In addition, a general-purpose reader rather than a dedicated reader can be used to detect post-hybridization biopolymers on the slide.

The present invention should be considered inclusive of the following alterations and modifications:

For example, liquid expansion based on piezoelectric devices or heaters can be used as means for discharging solutions, rather than using such rollers as referred to in the above-described embodiments.

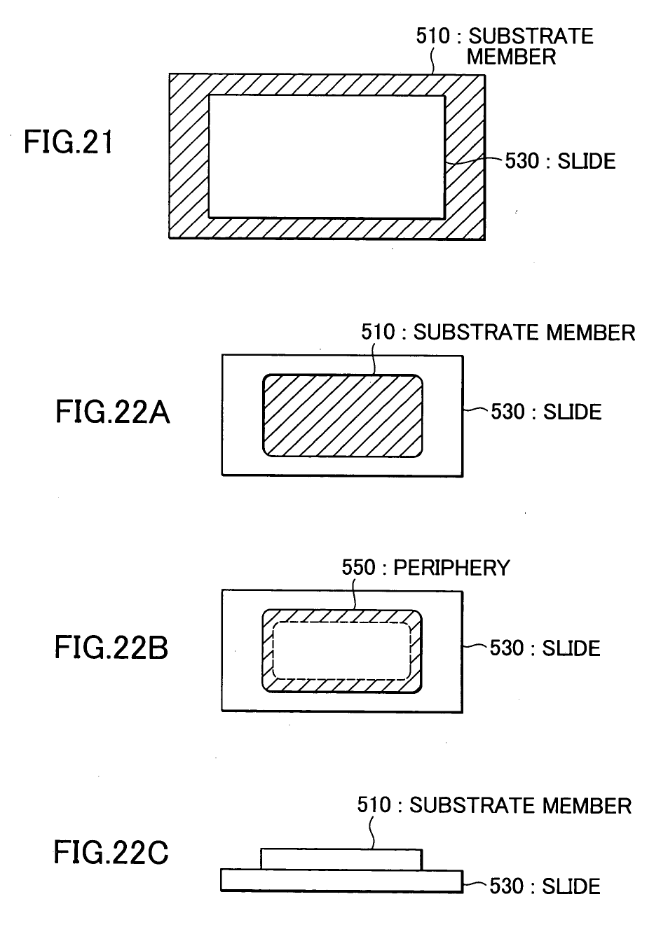

It is also possible to house the entirety of slide 530 within substrate member 510, as illustrated in FIG. 21. In contrast, slide 530 can be formed so that the entirety of substrate member 510 is seated upon slide 530, as illustrated in FIG. 22(a). In this case, however, the periphery 550 of substrate member 510 is removably adhered to the top surface of slide 530 using an adhesive agent, as illustrated in FIG. 22(b) and FIG. 22(c), without using cover 520.

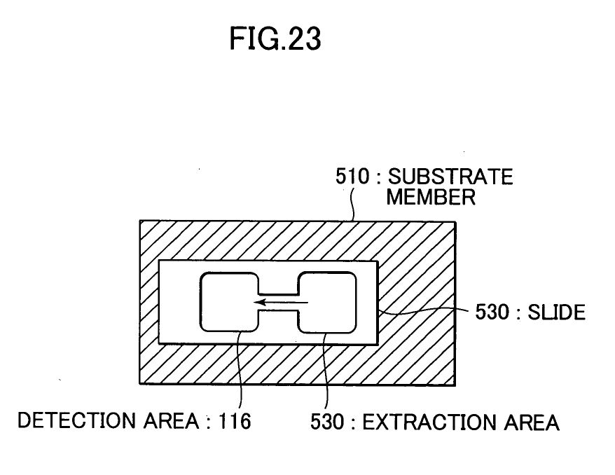

It is also possible to provide extraction area 519 for extracting DNA or RNA in the blood, in addition to detection area 116 for hybridization, on substrate member 510, as illustrated in FIG. 23, so that DNA or RNA can also be detected on slide 530.

Furthermore, labeling can be achieved by attaching a light-absorbing dye or luminescent dye, in addition to by attaching a fluorescent marker.

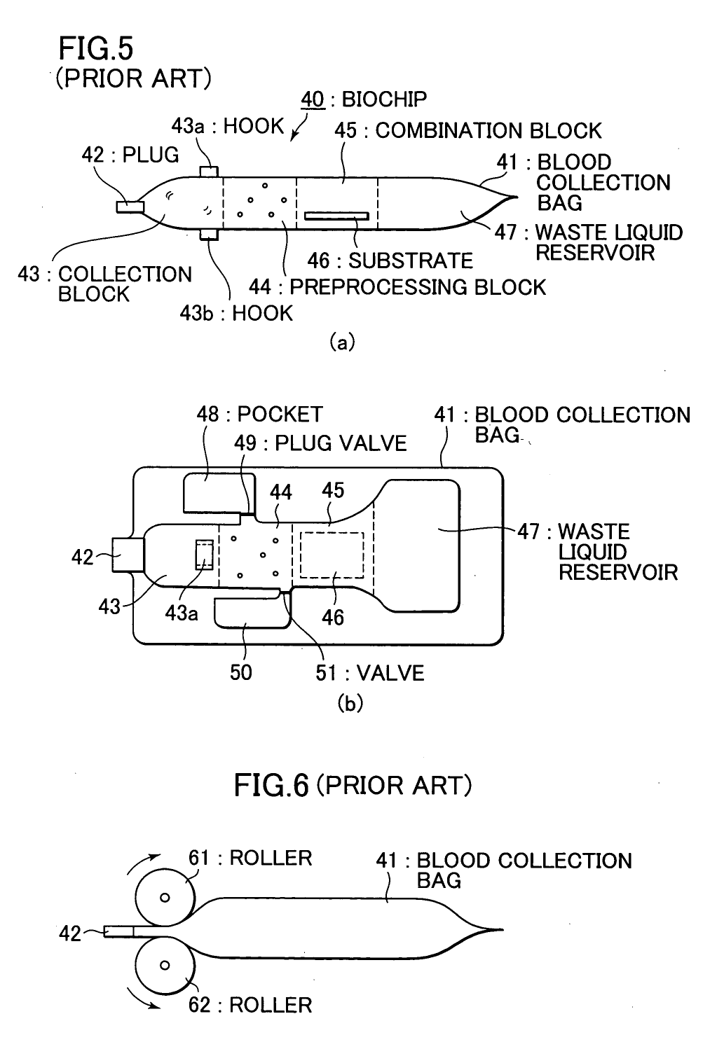

It is also possible to provide a preprocessing area in front of the connection, as indicated in the example of the conventional biochip cartridge illustrated in FIG. 5, and mix the preprocessing solution with the biological sample in that preprocessing area, in order to label biopolymers.

The biochip cartridge configured as described above provides the following advantageous effects:

(1) Preprocessing, such as labeling biopolymers, can be carried out within the biochip cartridge. In this case, there is no danger that viruses, for example, are released out of the biochip cartridge during processing since the sample is preprocessed within the airtightly sealed biochip cartridge.

(2) A general-purpose slide can be used for the biochip cartridge and biopolymers fixed (hybridized, for example) in the array area of the slide can easily be measured with a general-purpose reader, without the need for any dedicated reader.

What is claimed is:

1. A biochip cartridge comprising:
a tabular substrate member formed using an elastic material; and
a flexible cover airtightly attached to the surface of said substrate member;
wherein at least an area for storing biopolymers, an area for detecting desired biopolymers from said biopolymers that have been preprocessed, and a flow path for connecting said areas is formed on said substrate member, so that biopolymers can be successively moved from said biopolymer storage area to said biopolymer detection area through said flow path.

2. The biochip cartridge of claim 1, wherein a flexible cover is airtightly attached to the surface of said substrate member and a collection area for storing biopolymers, a preprocessing area for applying preprocessing to said biopolymers, a detection area for detecting biopolymers, from among said preprocessed biopolymers, that combine with previously prepared biopolymers and gaps serving as a flow path for connecting said collection area, said preprocessing area and said detection area are formed in and on said substrate member, so that biopolymers can be successively transferred from said collection area through said preprocessing area to said detection area.

3. The biochip cartridge of claim 1 or 2, wherein said biopolymers are transferred by pressing said cover with a roller-like rigid body and squeezing each gap formed in said substrate member from said collection area through said preprocessing area toward said detection area.

4. The biochip cartridge of claim 3, wherein a pocket to be filled with a preprocessing solution is formed in said substrate member and a preprocessing solution stored in said pocket is driven out into said preprocessing area when said roller is pressed down on said pocket.

5. The biochip cartridge of claim 3, wherein a waste liquid reservoir for storing waste liquid drained out of said detection area is formed in said substrate member.

6. The biochip cartridge of claim 1 or 2, wherein said cover is attached to both the top and bottom surfaces of said substrate member.

7. The biochip cartridge of claim 1 or 2, wherein gaps serving as said flow path formed in said substrate member are squeezed as said roller-like rigid body is pressed down on said gaps.

8. The biochip cartridge of claim 6, wherein said covers are formed using plastics or silica.

9. The biochip cartridge of claim 6, wherein said cover is formed using a transparent material so that optical detection can be achieved at least in said detection area.

10. The biochip cartridge of claim 4, wherein a plurality of said pockets for storing preprocessing solutions are formed in different positions so that when said substrate member is squeezed with said roller-like rigid body, a preprocessing solution is driven out of each of said pockets into said preprocessing area in a time-differentiated manner.

11. The biochip cartridge of claim 1 or 2, wherein said substrate member is formed into a wedge shape so that the thickness thereof gradually decreases from said collection area toward said detection area.

12. The biochip cartridge of claim 1 or 2, wherein a valve for checking the flow of solutions is provided in said flow path and said valve opens when a solution flowing through said flow path is pressurized.

13. The biochip cartridge of claim 1 or 2, wherein said substrate member is formed using a plastic-deformable material or gel.

14. The biochip cartridge of claim 1 or 2, wherein said biochip cartridge is made separable into a first housing for extracting and storing said biopolymers from a biological sample and a second housing having a joint for attachably and detachably coupling with said first housing to enable biopolymers to be injected from said first housing, so that biological samples can be injected into said first housing and transferred from said first housing said to second housing at different timings.

15. The biochip cartridge of claim 14, wherein said biopolymers are DNA, RNA such as mRNA or cDNA, or protein.

16. The biochip cartridge of claim 14, wherein said second housing is provided with a substrate on to which second biopolymers having sequences complementary to said biopolymers are fixed so that said second biopolymers are hybridized with biopolymers injected from said first housing.

17. The biochip cartridge of any of claim 14, wherein at least said first housing is formed using a material having good flexibility.

18. The biochip cartridge of claim 16, wherein said second housing is formed using a transparent material.

19. The biochip cartridge of claim 1 or 2, wherein a preprocessing mechanism for performing preprocessing in order to turn biological samples into measurable biopolymers is provided in said substrate member and a slide glass type biopolymer microarray is mounted on said biochip cartridge, so that said processed biopolymers can be fixed in the array area of said microarray.

20. The biochip cartridge of claim 19, wherein the short and long sides of said slide glass type biopolymer microarray are not greater than 25±1 mm and 75±1 mm, respectively.

21. The biochip cartridge of claim 19, wherein said preprocessing mechanism includes:
a collection area for storing biological samples;
a preprocessing solution storage for storing preprocessing solutions to be applied to said biological samples;
a washing solution storage for storing washing solutions used to clean post-preprocessing biopolymers;
a combination area for performing hybridization on said slide glass type biopolymer microarray;
a waste liquid reservoir for storing waste liquid; and
a flow path for connecting all of said areas and storages; so that biological samples can be successively transferred from said collection area through said preprocessing area to said detection area.

22. The biochip cartridge of claim 19, wherein said biological samples are transferred by squeezing said substrate member with a rigid roller in the direction from said collection area toward said combination area.

23. The biochip cartridge of claim 19, wherein said slide glass type biopolymer microarray is airtightly mounted on said substrate member in such a manner that the array area of said slide glass type biopolymer microarray is opposed to said combination area.

24. The biochip cartridge of claim 19, wherein a cover formed using a rigid material is attached to said substrate member and a cavity is formed therebetween, said slide glass type biopolymer microarray being airtightly mounted on said substrate member in such a manner that the array area of said slide glass type biopolymer microarray is opposed to said combination area.

25. The biochip cartridge of claim 19, wherein said preprocessing mechanism includes a mechanism for extracting DNA or RNA.
